(19) United States
(12) Patent Application Publication
(10) Pub. No.: US 2023/0204522 A1
(43) Pub. Date: Jun. 29, 2023

(54) RADIATION IMAGING APPARATUS, IMAGE PROCESSING APPARATUS, OPERATION METHOD FOR RADIATION IMAGING APPARATUS, AND NON-TRANSITORY COMPUTER READABLE STORAGE MEDIUM

(71) Applicant: CANON KABUSHIKI KAISHA, Tokyo (JP)

(72) Inventors: Takashi Takasaki, Kanagawa (JP); Toshiharu Sumiya, Kanagawa (JP); Akiya Nakayama, Kanagawa (JP); Ritsuya Tomita, Kanagawa (JP)

(21) Appl. No.: 18/052,524

(22) Filed: Nov. 3, 2022

(30) Foreign Application Priority Data
Nov. 10, 2021 (JP) 2021-183448

(51) Int. Cl. G01N 23/04; G01N 23/06; H04N 25/30

(52) U.S. Cl. CPC G01N 23/04; G01N 23/06; H04N 25/30; G01N 2223/401

(57) ABSTRACT

A radiation imaging apparatus includes a detection unit configured to detect radiation emitted from a radiation irradiation unit, the apparatus comprises a processing unit configured to obtain dose distribution information regarding the radiation with which the detection unit is irradiated from the radiation irradiation unit. The processing unit corrects, using the dose distribution information, an image signal output from the detection unit.

### BACKGROUND

### Field of Disclosure

The present disclosure generally relates to apparatuses and corresponding processes involving the generation or use of electromagnetic radiation, and more specifically it relates to a radiation imaging apparatus, an image processing apparatus, an operation method for the radiation imaging apparatus, and a non-transitory computer readable storage medium.

### Description of Related Art

As an imaging apparatus used for non-destructive inspection or medical imaging diagnosis by radiation, there is a radiation imaging apparatus using a flat panel detector (to be referred to as an FPD hereinafter) made of a semiconductor material, in which the position of the FPD is variable. In such radiation imaging apparatus, the dose of radiation that reaches the FPD may change due to the heel effect, and a difference in effective thickness of an added radiation filter in accordance with the position of the FPD.

Japanese Patent Laid-Open No. 2009-171990 discloses, as a technique for correcting a change in an output signal of an FPD caused by a change in dose of radiation that reaches the FPD, a technique for saving, in advance, distribution information regarding radiation emitted from a radiation irradiation apparatus as two-dimensional distribution information on a plane on which the FPD moves, and correcting an output signal of the FPD based on the saved two-dimensional distribution information.

However, in the radiation imaging apparatus, if, for example, the distance between the radiation irradiation apparatus and the FPD (source-to-image distance or SID) is changed, the dose of radiation that reaches the FPD changes in accordance with the relative distance relationship (the radiation intensity is inversely proportional to the square of the distance). Therefore, for example, even if radiation dose distribution information obtained by an FPD located at a given distance from the radiation irradiation apparatus is used to correct an output signal of an FPD located at a different distance, an image signal output from the FPD may be non-uniform due to a change in radiation dose distribution.

### SUMMARY

The present disclosure reduces the variation of an image signal that may be caused in accordance with a three-dimensional positional relationship between the radiation irradiation apparatus and the detector.

According to one aspect of the present disclosure, there is provided a radiation imaging apparatus including a detection unit configured to detect radiation emitted from a radiation irradiation unit, the apparatus comprising a processing unit configured to obtain dose distribution information regarding the radiation with which the detection unit is irradiated from the radiation irradiation unit, wherein the processing unit corrects, using the dose distribution information, an image signal output from the detection unit.

Further features of the present disclosure will become apparent from the following description of exemplary embodiments (with reference to the attached drawings).

### BRIEF DESCRIPTION OF THE DRAWINGS

FIG. 1 is a view exemplifying the overall arrangement of a radiation imaging apparatus according to the first embodiment;

FIG. 2 is a flowchart for explaining the procedure of processing of generating dose distribution information according to the first embodiment;

FIG. 3 is a table exemplifying a table holding imaging conditions;

FIG. 4 is a view for explaining the processing of generating dose distribution information according to the first embodiment;

FIG. 5 is a flowchart for explaining the procedure of an operation method for the radiation imaging apparatus according to the first embodiment;

FIG. 6 is a view for explaining processing of correcting an image signal according to the first embodiment;

FIG. 7 is a view exemplifying the positional relationship between a radiation irradiation unit and a radiation detector in processing according to the second embodiment;

FIG. 8 is a view exemplifying the positional relationship between a radiation irradiation unit and a radiation detector in processing according to the third embodiment;

FIG. 9 is a view showing an example of display of a region not to be used in imaging according to the fourth embodiment; and

FIG. 10 is a view exemplifying the positional relationship between a radiation irradiation unit and a radiation detector in processing according to the fourth embodiment.

### DESCRIPTION OF THE EMBODIMENTS

Hereinafter, embodiments will be described in detail with reference to the attached drawings. Note, the following embodiments are not intended to limit the scope of the claimed subject matter. Multiple features are described in the embodiments, but limitation is not made to any particular embodiment that requires all such features, and multiple such features of different embodiments may be combined or interchanged as appropriate. Furthermore, in the attached drawings, the same reference numerals are given to the same or similar configurations, and redundant description thereof is omitted.

In the following embodiments and claims, radiation refers to electromagnetic radiation including, but not limited to, X-rays, α-rays, β-rays, γ-rays, and various kinds of particle rays, and is applicable to a radiation imaging apparatus for capturing a radiation image of an object.

### First Embodiment

(Arrangement of Radiation Imaging Apparatus)

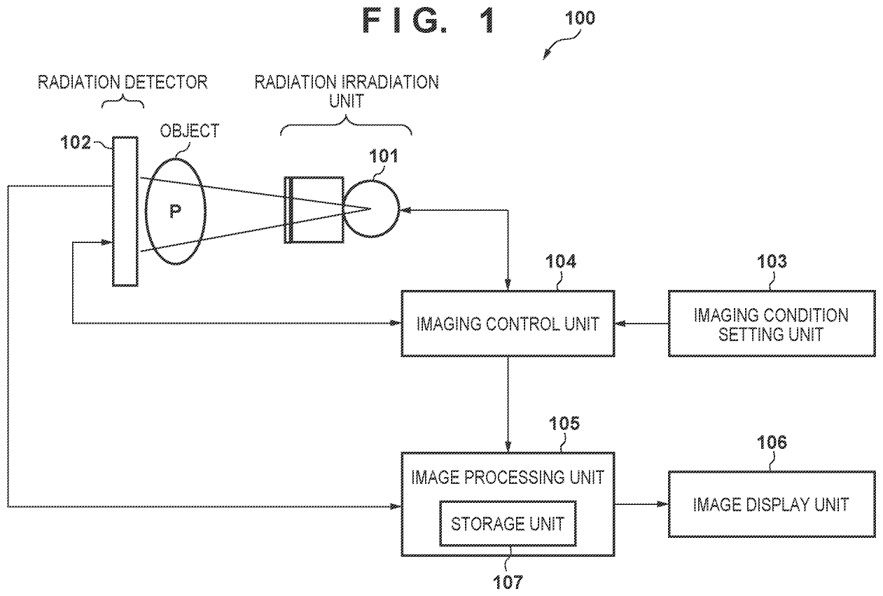

A radiation imaging apparatus 100 according to the first embodiment will be described. FIG. 1 is a view exemplifying the overall functional arrangement of the radiation imaging apparatus 100 according to the first embodiment. Referring to FIG. 1, a radiation irradiation unit 101 irradiates an object P with radiation. The radiation irradiation unit 101 includes a radiation generator (tube bulb) that generates radiation, a collimator that defines the beam divergence angle of the radiation generated by the radiation generator, an aluminum filter attachable/detachable to/from the collimator, and a filter replacement mechanism. For the radiation imaging apparatus 100 according to this embodiment, a plurality of kinds of aluminum filters having different thicknesses of, for example, 2 mm, 5 mm, and the like can be used as the aluminum filter (to also be referred to as the AL filter hereinafter). The filter replacement mechanism can perform a filter replacement operation of attaching or detaching an aluminum filter to or from the collimator.

The radiation imaging apparatus 100 includes a radiation detector 102 that detects radiation emitted from the radiation irradiation unit 101. The radiation detector 102 is a flat panel detector (FPD) in which pixels each including an image sensor for outputting a radiation signal corresponding to radiation (incident light) emitted from the radiation irradiation unit 101 are arranged in an array (a region of a two-dimensional plane). The photoelectric converting element of each pixel converts, into a radiation signal (to also be referred to as an image signal hereinafter) as an electrical signal, light converted by a phosphor, and the capacitor of each pixel accumulates the charges of the radiation signal (image signal). The radiation detector 102 reads out the image signal accumulated in the capacitor of each pixel, and transmits it to an image processing unit 105.

An imaging condition setting unit 103 includes an imaging condition input unit (for example, an input unit not shown) used by an operator to input imaging condition information such as a tube voltage, a tube current, an irradiation time, a focal size, the thickness of an aluminum filter to be added, distance information between the radiation irradiation unit 101 and the radiation detector 102, and the like. The imaging condition setting unit 103 transmits, to an imaging control unit 104, the imaging condition information input from the imaging condition input unit. Examples of the imaging condition input unit include, but are not limited to, well-known inputting devices used to provide data and control signals to a processing system. Such inputting devices may include one or more of a keyboard and a mouse, a touchscreen, a microphone to input by voice command, and the like.

The imaging control unit 104 can control the radiation irradiation unit 101 and the radiation detector 102 based on the imaging condition information received from the imaging condition setting unit 103 and the image signal received from the radiation detector 102.

The imaging control unit 104 can generate, based on the imaging condition information obtained from the imaging condition setting unit 103, an irradiation instruction signal and an imaging control signal. The irradiation instruction signal is for causing the radiation irradiation unit 101 to perform radiation irradiation; and the imaging control signal is for driving the radiation detector 102. The imaging control unit 104 can also control the radiation irradiation timing of the radiation irradiation unit 101 and the imaging timing of the radiation detector 102.

Furthermore, the imaging control unit 104 controls a position control mechanism (not shown) for controlling the relative position between the radiation irradiation unit 101 and the radiation detector 102. The imaging control unit 104 can control the operation of the position control mechanism to align the relative position with a predetermined position based on the imaging condition information. Examples of the position control mechanism include, but are not limited to, well-known positioning stages used to hold the radiation detector 102.

An image processing apparatus according to this embodiment includes the image processing unit 105 that corrects, using three-dimensional dose distribution information regarding the radiation emitted from the radiation irradiation unit 101, the image signal output from the radiation detector 102 that detects the radiation. The image processing unit 105 may include, for example, a dedicated graphics processing unit (GPU) card which applies image processing such as gradation processing and noise reduction processing to radiation image data. The radiation image data is based on the image signal received from the radiation detector 102. The image processing unit 105 transmits, to an image display unit 106, the signal having undergone the image processing. The image display unit 106 functions as a display control unit to convert the signal obtained from the image processing unit 105 into a two-dimensional image (radiation image data) and output (display) the converted two-dimensional image (radiation image data) to a display device such as a liquid crystal display (LCD) or an organic light emitting diode (OLED) display monitor. This allows the operator to confirm the radiation image data obtained by imaging an imaging portion of the object. The image processing unit 105 according to this embodiment obtains three-dimensional dose distribution information regarding the radiation with which the radiation detector 102 is irradiated from the radiation irradiation unit 101, and corrects, using the obtained dose distribution information, the image signal output from the radiation detector 102. Processing of generating dose distribution information will be described in detail below.

### (Generation of Three-Dimensional Dose Distribution Information)

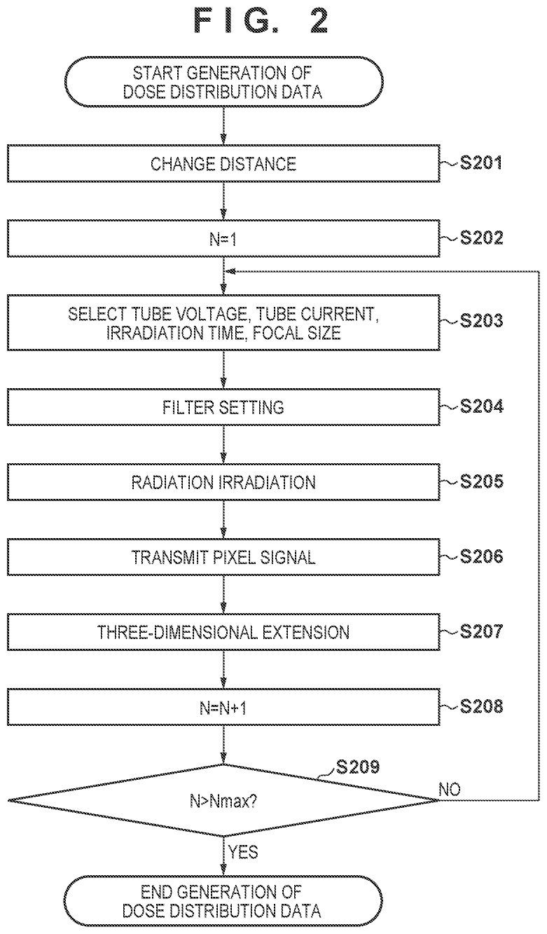

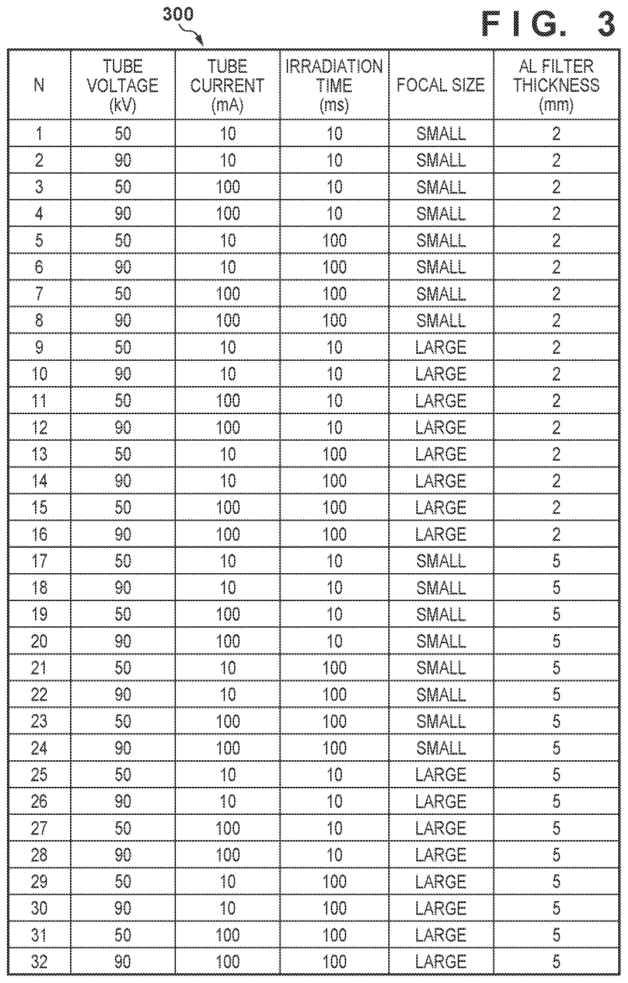

FIG. 2 is a flowchart for explaining the procedure of the processing of generating the three-dimensional dose distribution information of the radiation dose. FIG. 3 is a table exemplifying a table 300 that holds imaging conditions to be used in the processing of generating the three-dimensional dose distribution information. The table 300 is assigned with an imaging condition number N. If the imaging control unit 104 selects one of the numbers in the table 300, the imaging condition corresponding to the imaging condition number N is set. At this time, the imaging control unit 104 or the imaging condition setting unit 103 can hold the table 300 in an internal storage unit, or can hold the table 300 in an external server and obtain the imaging condition by communication via a network.

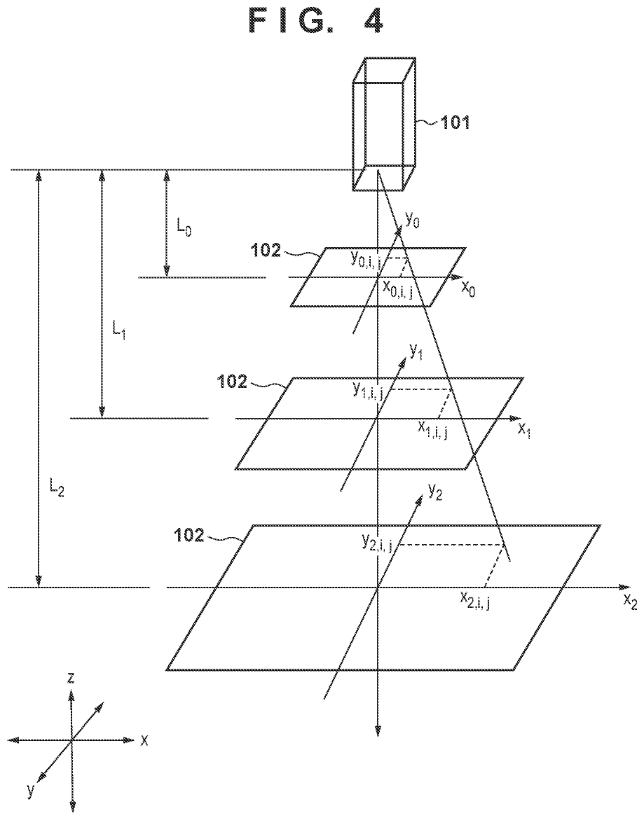

FIG. 4 is a view exemplifying the positional relationship between the radiation irradiation unit 101 and the radiation detector 102 in the processing of generating the three-dimensional dose distribution information. Referring to FIG. 4, reference symbols L₀, L₁, and L₂ each denote the distance in the vertical direction (z axis) between the radiation irradiation unit 101 and the radiation detector 102. The distance L₀ represents the minimum value within a settable distance range.

As will be described in detail with reference to the processing procedure shown in FIG. 2, in this embodiment, the image processing unit 105 obtains (estimates) a three-dimensional image signal D_{Li} based on the relationship between the distance L₀ and the different distance (L₁ or L₂) with reference to a three-dimensional image signal Do obtained on a plane at the distance L₀. Note that FIG. 4 exemplifies the distances L₁ and L₂ but the present disclosure is not limited to this example. With respect to a plurality of distances, pieces of three-dimensional dose distribution information can be generated and stored in a database for any number of distances. This can correct the image signal in accordance with the various imaging conditions including the distance when performing imaging in accordance with the physical constitution of the object and the imaging portion.

The image processing unit 105 according to this embodiment obtains dose distribution information using the distance relationship between the radiation detector 102 and the radiation irradiation unit 101. That is, the image processing unit 105 obtains three-dimensional dose distribution information regarding the radiation emitted from the radiation irradiation unit 101 using the relationship of the square of the distance ratio between the reference distance (L₀) of the dose distribution and the distance (L₁ or L₂) between the radiation detector 102 and the radiation irradiation unit 101.

By using, as reference information, the image signal Do on the plane at the distance L₀, an image signal Dᵢ on a plane at a different distance (Lᵢ) can be a value obtained by multiplying (L₀/Lᵢ)² by the image signal D₀ in accordance with the three-dimensional distance relationship (the inverse square law of distance). Processing using practical formulas will be described in detail later.

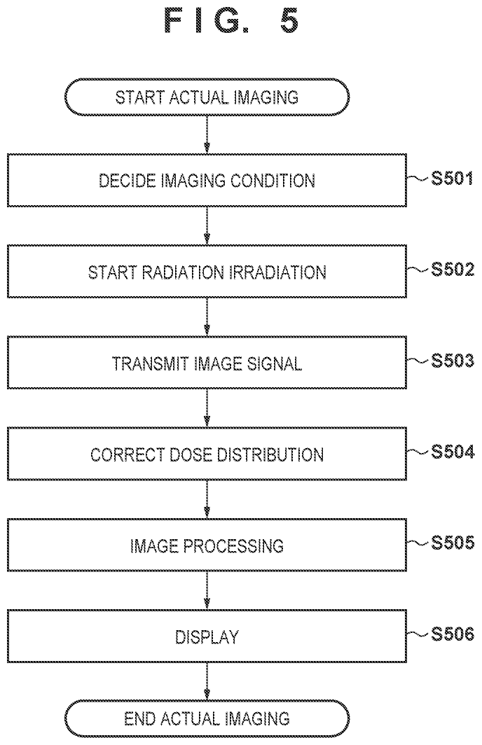

In the processing procedure shown in FIG. 2 to be described below, the processing of generating the three-dimensional dose distribution information will be explained. The processing procedure of actual imaging (radiation imaging of the object) using the three-dimensional dose distribution information will be described with reference to FIG. 5.

If the operator presses a processing start button provided in the imaging condition setting unit 103, the processing shown in FIG. 2 starts.

In step S201, the imaging control unit 104 controls the position control mechanism to execute such control that the distance between the radiation irradiation unit 101 and the radiation detector 102 is set to the minimum value L₀ (FIG. 4) within the settable distance range.

In step S202, the imaging control unit 104 sets an initial value of 1 in the imaging condition number N.

In step S203, the imaging control unit 104 selects the imaging condition corresponding to the imaging condition number N from the imaging condition table 300 exemplified in FIG. 3. At this time, the imaging condition table 300 shown in FIG. 3 holds an imaging condition including a tube voltage (kV), a tube current (mA), an irradiation time (ms), a focal size, and an AL filter thickness. The imaging control unit 104 selects (obtains) information of the imaging condition (tube voltage (kV), tube current (mA), irradiation time (ms), focal size, and AL filter thickness) corresponding to the imaging condition number N with reference to the table 300 shown in FIG. 3. The imaging control unit 104 transmits the selected information (imaging condition information) to the radiation irradiation unit 101.

In step S204, the radiation irradiation unit 101 attaches the aluminum filter (radiation filtration filter) matching the aluminum filter thickness information to the collimator by the incorporated filter replacement mechanism based on the imaging condition information received from the imaging control unit 104.

In step S205, the imaging control unit 104 transmits an irradiation instruction signal for instructing radiation irradiation to the radiation irradiation unit 101. Upon receiving the irradiation instruction signal from the imaging control unit 104, the radiation irradiation unit 101 irradiates the radiation detector 102 with the radiation. Furthermore, the imaging control unit 104 generates an imaging control signal for driving the radiation detector 102, and transmits the generated imaging control signal to the radiation detector 102. The radiation detector 102 converts the arrived radiation into an image signal for each pixel based on the imaging control signal received from the imaging control unit 104. The photoelectric converting element of each pixel accumulates the converted image signal (charges) in the capacitor of each pixel.

In step S206, the output unit of the radiation detector 102 reads out the signal from each pixel based on the imaging control signal, and transmits the image signal for each pixel to the image processing unit 105. That is, the output unit of the radiation detector 102 transmits the image signal accumulated in the capacitor of each pixel to the image processing unit 105 based on the imaging control signal.

In step S207, the image processing unit 105 generates three-dimensional dose distribution information of the dose of the radiation emitted from the radiation irradiation unit 101, using the image signal for each pixel on the plane at the distance L₀, which has been received from the radiation detector 102.

D_{0,i,j}(X_{0,i,j}y_{0,i,j},L₀) (a)

where D_{0,i,j} represents an image signal (dose distribution information) for each pixel of the radiation detector 102 on the plane at the distance L₀, which has been received by the image processing unit 105 in step S206. Subscripts i and j represent the pixel coordinates (pixel positions) of the radiation detector 102 on the X-axis and the Y-axis, respectively.

In step S207, with respect to all the pixels, the image processing unit 105 converts the image signal D_{0,i,j} for each pixel detected on the plane at the distance L₀ into an image signal D_{1,i,j} for each pixel on the plane at the distance L₁ different from the distance L₀ by equation (b) below based on the three-dimensional distance relationship (the inverse square law of distance) using the ratio of the distances between the radiation irradiation unit 101 and the radiation detector 102.

$$\begin{matrix}{D}_{1 , i , j} ( {x}_{1 , i , j} , {y}_{1 , i , j} , {L}_{1} ) = \frac{( {x}_{0 , i , j}^{2} + {y}_{0 , i , j}^{2} + {L}_{0}^{2} )}{( {x}_{1 , i , j}^{2} + {y}_{1 , i , j}^{2} + {L}_{1}^{2} )} \times {D}_{0 , i , j} ( {x}_{0 , i , j} , {y}_{0 , i , j} , {L}_{0} ) \\ = \frac{( {x}_{0 , i , j}^{2} + {y}_{0 , i , j}^{2} + {L}_{0}^{2} )}{\{ {( \frac{{L}_{1}}{{L}_{0}} \cdot {x}_{0 , i , j} )}^{2} + {( \frac{{L}_{1}}{{L}_{0}} \cdot {y}_{0 , i , j} )}^{2} + {L}_{1}^{2} \}} \times \\ {D}_{0 , i , j} ( {x}_{0 , i , j} , {y}_{0 , i , j} , {L}_{0} ) \\ = \frac{{L}_{0}^{2}}{{L}_{1}^{2}} \times {D}_{0 , i , j} ( {x}_{0 , i , j} , {y}_{0 , i , j} , {L}_{0} )\end{matrix} \qquad ( b )$$

With respect to all the pixels, the image processing unit 105 approximates, to a quadratic function, the converted image signal D_{1,i,j} for each pixel on the plane at the distance Lᵢ by equation (c) below. At this time, the image processing unit 105 decides each coefficient of the quadratic function by the least square method. Equation (c) indicates the dose distribution information on the plane at the distance L₁.

D₁(x₁,y₁,L₁)=a₁,x₁²+b₁x₁+c₁y₁²+d₁y₁+e₁ (c)

Similar to the case of the plane at the distance L₁, with respect to all the pixels, the image processing unit 105 converts the image signal D_{0,i,j} for each pixel detected on the plane at the distance L₀ into an image signal D_{2,i,j} for each pixel on the plane at the distance L₂ different from the distances L₀ and L₁ by equation (d) below based on the three-dimensional distance relationship (the square law of the distance) using the ratio of the distances between the radiation irradiation unit 101 and the radiation detector 102.

Then, with respect to all the pixels, the image processing unit 105 approximates, to a quadratic function, the converted image signal D_{2,i,j} for each pixel on the plane at the distance L₂ by equation (e) below. At this time, the image processing unit 105 decides each coefficient of the quadratic function by the least square method. Equation (e) indicates the dose distribution information on the plane at the distance L₂.

$$\begin{matrix}{D}_{2 , i , j} ( {x}_{2 , i , j} , {y}_{2 , i , j} , {L}_{1} ) = \frac{( {x}_{0 , i , j}^{2} + {y}_{0 , i , j}^{2} + {L}_{0}^{2} )}{( {x}_{2 , i , j}^{2} + {y}_{2 , i , j}^{2} + {L}_{2}^{2} )} \times {D}_{0 , i , j} ( {x}_{0 , i , j} , {y}_{0 , i , j} , {L}_{0} ) \\ = \frac{( {x}_{0 , i , j}^{2} + {y}_{0 , i , j}^{2} + {L}_{0}^{2} )}{\{ {( \frac{{L}_{2}}{{L}_{0}} \cdot {x}_{0 , i , j} )}^{2} + {( \frac{{L}_{2}}{{L}_{0}} \cdot {y}_{0 , i , j} )}^{2} + {L}_{2}^{2} \}} \times \\ {D}_{0 , i , j} ( {x}_{0 , i , j} , {y}_{0 , i , j} , {L}_{0} ) \\ = \frac{{L}_{0}^{2}}{{L}_{2}^{2}} \times {D}_{0 , i , j} ( {x}_{0 , i , j} , {y}_{0 , i , j} , {L}_{0} )\end{matrix} \qquad ( d )$$

$${D}_{2} ( {x}_{2} , {y}_{2} , {L}_{2} ) = {a}_{2} {x}_{2}^{2} + {b}_{2} {x}_{2} + {c}_{2} {y}_{2}^{2} + {d}_{2} {y}_{2} + {e}_{2} \qquad ( e )$$

The image processing unit 105 saves the dose distribution information in an internal storage unit 107 (memory). At this time, the pieces of decided coefficient information of the quadratic functions of equations (c) and (e) are saved in the storage unit 107 (memory). Note that the arrangement of the storage unit 107 is not limited to the storage unit inside the image processing unit 105, and the storage unit 107 may be provided in an external cloud server with which it is possible to perform communication via a network.

In step S208, the imaging control unit 104 increments the imaging condition number N by one.

In step S209, the imaging control unit 104 determines whether the imaging condition number N exceeds a preset upper limit threshold Nₘₐₓ of the imaging condition number. If the imaging condition number N does not exceed the upper limit threshold Nₘₐₓ of the imaging condition number (NO in step S209), the imaging control unit 104 returns the process to step S203, and similarly repeats the processes in steps S203 to S208.

On the other hand, if it is determined in step S209 that the imaging condition number N is equal to or larger than the upper limit threshold Nₘₐₓ of the imaging condition number (YES in step S209), the imaging control unit 104 ends the processing of generating the dose distribution information. With this processing, the pieces of coefficient information of the quadratic functions corresponding to equations (c) and (e) are saved in the storage unit 107 with respect to all the conditions (N=1 to Nₘₐₓ) set in the imaging condition table 300. In one example, Nₘₐₓ can be set to N=32 as shown in the example of table 300 of FIG. 3.

### (Procedure of Operation Method for Radiation Imaging Apparatus)

FIG. 5 is a flowchart for explaining the procedure of the operation method for the radiation imaging apparatus according to the first embodiment. The image processing unit 105 uses the three-dimensional dose distribution information obtained by the processing procedure shown in FIG. 2 to perform correction (gain correction) for the image signal output from the output unit of the radiation detector 102 by radiation imaging of the object P.

In step S501, the operator selects one imaging condition from the imaging condition table 300 shown in FIG. 3 and inputs the corresponding imaging condition number N by the imaging condition input unit (not shown) provided in the imaging condition setting unit 103. Furthermore, the operator uses the imaging condition input unit to select and input one of the distances L₁ and L₂ between the radiation irradiation unit 101 and the radiation detector 102, which is provided in the imaging condition setting unit 103.

The operator inputs the plane position of the radiation detector 102 (the pixel coordinates (pixel position) of the radiation detector 102 on the X-axis and the Y-axis) by the plane position input unit of the radiation detector 102 provided in the imaging condition setting unit 103. If the information is input, the imaging condition setting unit 103 transmits, to the imaging control unit 104, the input imaging condition information corresponding to the imaging condition number N, the input distance information between the radiation irradiation unit 101 and the radiation detector 102, and the input plane position information (pixel coordinates (pixel position) of the radiation detector 102.

In step S502, the imaging control unit 104 controls the position control mechanism based on the distance information and plane position information received from the imaging condition setting unit 103, thereby controlling the distance between the radiation irradiation unit 101 and the radiation detector 102, and the plane position of the radiation detector 102.

Next, the imaging control unit 104 transmits, to the radiation irradiation unit 101, the imaging condition information corresponding to the imaging condition number N received from the imaging condition setting unit 103. In the radiation irradiation unit 101, the imaging condition (tube voltage (kV), tube current (mA), irradiation time (ms), focal size, and AL filter thickness) and the like are set based on the information received from the imaging control unit 104.

Next, the imaging control unit 104 generates an irradiation instruction signal for instructing radiation irradiation, and transmits the generated irradiation instruction signal to the radiation irradiation unit 101. Upon receiving the irradiation instruction signal, the radiation irradiation unit 101 irradiates the object P with the radiation under the imaging condition set based on the imaging condition number. Furthermore, the imaging control unit 104 generates an imaging control signal for driving the radiation detector 102, and transmits the generated imaging control signal to the radiation detector 102. Then, the radiation detector 102 converts the arrived radiation into an image signal for each pixel based on the received imaging control signal. The photoelectric converting element of each pixel accumulates the converted image signal (charges) in the capacitor of each pixel.

In step S503, the radiation detector 102 transmits the image signal for each pixel to the image processing unit 105. The output unit of the radiation detector 102 reads out the signal from each pixel based on the imaging control signal, and transmits the image signal for each pixel to the image processing unit 105.

In step S504, the image processing unit 105 performs, for the image signal for each pixel received in step S503, processing of correcting the variation of the image signal caused by the three-dimensional dose distribution regarding the radiation emitted from the radiation irradiation unit 101, that is, the variation of the image signal that may be caused in accordance with the three-dimensional distance relationship between the radiation irradiation unit 101 and the radiation detector 102.

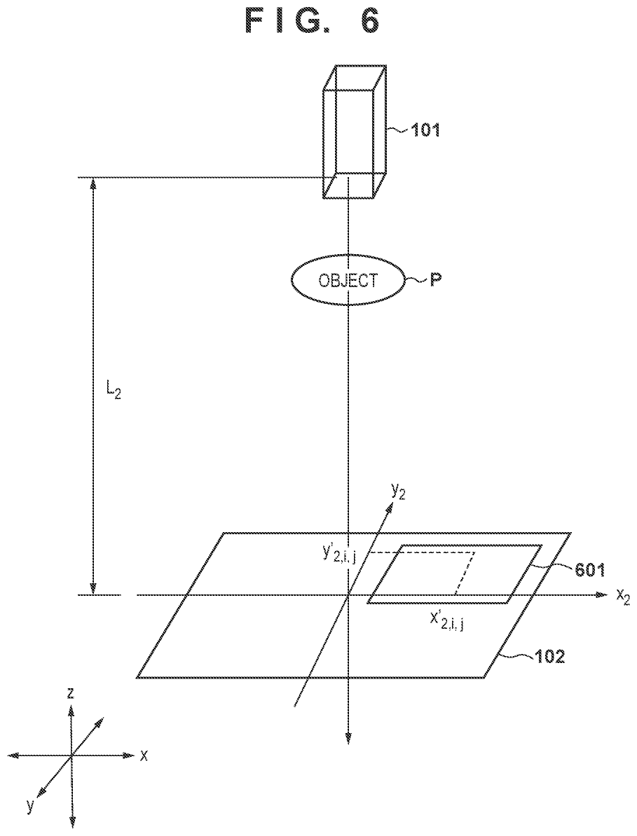

FIG. 6 is a view for explaining the processing of correcting, using the three-dimensional dose distribution information, the image signal obtained by radiation imaging of the object P. Referring to FIG. 6, the distance L₂ indicates the distance in the vertical direction between the radiation irradiation unit 101 and the radiation detector 102, which has been set in step S501. An effective pixel area 601 shown in FIG. 6 is a pixel region for generating an image signal based on radiation with which the radiation incident surface of the radiation detector 102 is irradiated, and can be set based on the plane position information of the radiation detector 102, which has been set in step S501.

With respect to all the pixels, the image processing unit 105 divides an image signal S_{2,i,j} for each pixel, which has been received in step S503, by the dose distribution information at the corresponding pixel position, as given by equation (f) below. At this time, the coefficients of the quadratic function decided in step S207 are used. The image signal S_{2,i,j} for each pixel received in step S503 includes the variation of the image signal caused by the three-dimensional dose distribution regarding the radiation emitted from the radiation irradiation unit 101, that is, the variation of the image signal that may be caused in accordance with the three-dimensional distance relationship between the radiation irradiation unit 101 and the radiation detector 102. By dividing the image signal S_{2,i,j} by the three-dimensional dose distribution information, the image signal (output signal) of each pixel of the radiation detector 102 is corrected based on the three-dimensional distance relationship between the radiation irradiation unit 101 and the radiation detector 102.

By correcting the image signal (output signal) of each pixel of the radiation detector 102, it is possible to reduce the variation of the image signal that may be caused in accordance with the three-dimensional positional relationship between the radiation irradiation unit 101 and the radiation detector 102, and suppress non-uniformity of an image that may be caused by the three-dimensional dose distribution regarding the radiation.

$${P}_{2 , i , j} ( {x}_{2 , i , j}^{′} , {y}_{2 , i , j}^{′} , {L}_{2} ) = \frac{{S}_{2 , i , j} ( {x}_{2 , i , j}^{′} , {y}_{2 , i , j}^{′} , {L}_{2} )}{{a}_{2} {x}_{2 , i , j}^{′2} + {b}_{2} {x}_{2 , i , j}^{′} + {c}_{2} {y}_{2 , i , j}^{′ 2} + {d}_{2} {y}_{2 , i , j}^{′} + {e}_{2}} \qquad ( f )$$

In step S505, the image processing unit 105 performs gradation processing, noise reduction processing, and the like for a gain-corrected image signal P_{2,i,j} after the division operation, which has been obtained in step S504. Then, the image processing unit 105 transmits, to the image display unit 106, the signal having undergone the image processing such as gradation processing and noise reduction processing.

In step S506, the image display unit 106 converts the received signal into a two-dimensional image, and outputs (displays) the converted two-dimensional image (radiation image data) on the display device such as a monitor. Then, the processing of imaging the object ends.

### (Modification)

In the first embodiment, the dose distribution information on the plane at the distance L₁ or L₂ is approximated by the quadratic function indicated by equation (c) or (d). The approximation method is not limited to the quadratic function, and a higher-order function or the like may be used. In the first embodiment, with respect to the dose distribution information on the plane at the distance L₁ or L₂, the dose distribution information on the entire surface (radiation incident surface) of the detection unit is approximated by the quadratic function. The radiation incident surface of the detection unit may be divided into a plurality of regions, and dose distribution information for each divided region may be approximated, and saved in the storage unit 107 for each region. That is, the image processing unit 105 may obtain the dose distribution information for each of the plurality of regions obtained by dividing the detection plane of the radiation detector 102, save the dose distribution information obtained for each region in the storage unit 107, and correct, using the dose distribution information for each region, the image signal output from the radiation detector 102.

Furthermore, in the first embodiment, the dose distribution information on the plane at the distance L₁ or L₂ is calculated only from the dose distribution information obtained on the plane at the distance L₀ as the reference distance. However, a plurality of distances L₀₁, L₀₂, . . . , L₀ₙ may be set as reference distances, and dose distribution information may be obtained for each distance. Then, in accordance with the distance between the radiation irradiation unit 101 and the radiation detector 102 at the time of imaging the object, the weighted average of the values of the pieces of dose distribution information for the respective distances may be used. That is, the image processing unit 105 may weight, in accordance with the distances, the plurality of pieces of distribution information calculated using the plurality of reference distances (distances L₀₁, L₀₂, . . . , L₀ₙ) of the dose distribution, and average them, thereby obtaining dose distribution information.

If there are provided a plurality of radiation detectors 102 and the radiation detector 102 is switched in accordance with imaging, dose distribution information calculated by a given radiation detector 102 may be used to perform the correction processing in step S504 for an image signal obtained by another radiation detector 102.

If the plurality of radiation detectors 102 are used to perform radiation imaging, they can share dose distribution information. That is, the image processing unit 105 may use dose distribution information obtained based on an image signal of the radiation detector 102 to correct an image signal output from another different radiation detector 102. Thus, if dose distribution information calculated by a given radiation detector 102 is generated, it is possible to perform radiation imaging of the object P (FIG. 6) without performing the processing (the processing procedure shown in FIG. 2) of generating the dose distribution information in another radiation detector 102, and to improve the throughput of the imaging processing while reducing the load of the operator.

Furthermore, in the first embodiment, at the time of performing the imaging procedure, among the pixel signals of the radiation detector 102, the pixel signals of a through-exposure portion where the radiation emitted from the radiation irradiation unit 101 arrives without being transmitted through the object P may be used to calculate three-dimensional dose distribution information regarding the radiation emitted from the radiation irradiation unit 101. For example, the image processing technique can be used to analyze and divide the radiation image into an object portion where the radiation is transmitted through the object and a through-exposure portion where the radiation directly arrives the radiation detector 102 without being transmitted through the object. If the type of the object P and the like are known and only the same object is imaged, it is possible to predict a through-exposure portion based on the geometric arrangement. It is also possible to estimate a through-exposure portion by obtaining the position of the object P with respect to the radiation detector 102 using a visible light camera. The image processing unit 105 according to this embodiment can also specify, from the image signals at the time of imaging the object, a through-exposure region as a region where the radiation detector 102 is directly irradiated with the radiation, and obtain (correct) the dose distribution information using the image signals of the through-exposure region.

The coefficients of the quadratic function for approximating the dose distribution may be corrected in accordance with a change in sensitivity of the pixel. For example, at the time of performing the second or subsequent actual imaging procedure, if irradiation with strong radiation is performed in previous imaging, and the sensitivity of the pixel of the radiation detector 102 changes to exceed a predetermined threshold, the function (the coefficients of the quadratic function) for approximating the dose distribution information may be corrected to reduce the change in sensitivity. If the image signal output from each pixel of the radiation detector 102 changes to exceed the threshold, the image processing unit 105 according to this embodiment can obtain (correct) the dose distribution information using the image signal for which the sensitivity has been corrected to be equal to or smaller than the threshold.

According to this embodiment, it is possible to reduce the variation of an image signal that may be caused in accordance with the three-dimensional positional relationship between the radiation irradiation unit 101 and the radiation detector 102, and suppress non-uniformity of an image that may be caused by the three-dimensional dose distribution regarding the radiation.

### Second Embodiment

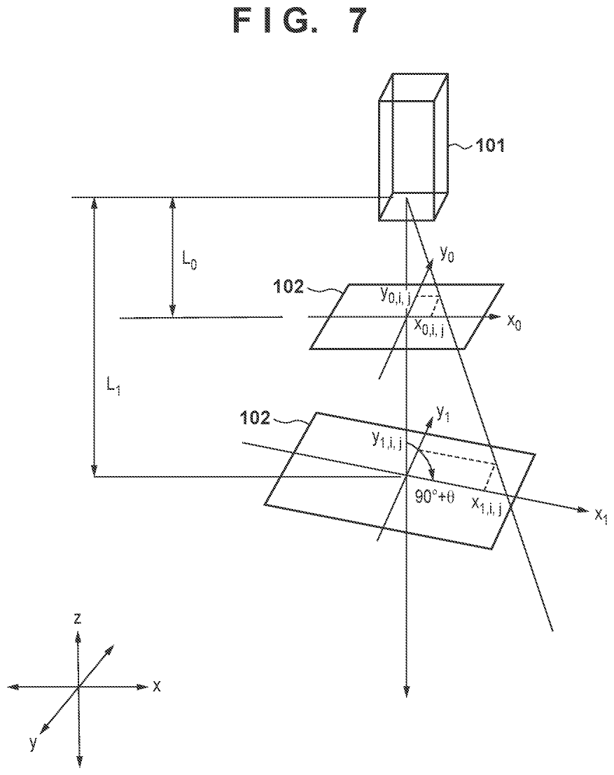

The second embodiment of the present disclosure will be described next. This embodiment is different from the first embodiment in that a relative angle deviation between a radiation irradiation unit 101 and a radiation detector 102 is measured, and the variation of an image signal caused in accordance with the three-dimensional positional relationship including the angle deviation is reduced. The same description as in the first embodiment will be omitted, and only the difference from the first embodiment will be described below. Processing of generating three-dimensional dose distribution information of a radiation dose according to the second embodiment will be described with reference to FIG. 7.

FIG. 7 is a view exemplifying the positional relationship between the radiation irradiation unit 101 and the radiation detector 102 in the processing of generating the three-dimensional dose distribution information. In the second embodiment, a case in which the irradiation center axis of the radiation irradiation unit 101 and the plane of the radiation detector 102 are deviated from the vertical direction by an angle θ with respect to the X-axis direction at the time of imaging an object, as shown in FIG. 7, will be described. The radiation detector 102 incorporates an angle detector capable of detecting a three-dimensional angle, and the angle θ is measured by the angle detector incorporated in the radiation detector 102. The radiation detector 102 transmits information of the angle θ measured by the angle detector to an image processing unit 105. In the second embodiment, the operator can set the distance between the radiation irradiation unit 101 and the radiation detector 102 to an arbitrary position between distances L₀ and L₂. In this embodiment, a case in which a distance L₁ is set, as shown in FIG. 7, will be described.

The image processing unit 105 according to this embodiment obtains (corrects) three-dimensional dose distribution information using the angle relationship between the radiation detector 102 and the radiation irradiation unit 101, which has been detected at the time of imaging the object, and corrects an image signal output from the radiation detector 102 using the obtained (corrected) three-dimensional dose distribution information.

In the second embodiment, the three-dimensional extension processing in step S207 of FIG. 2 according to the first embodiment is performed in the dose distribution correction processing in step S504 of FIG. 5 according to the first embodiment. In the second embodiment, the image processing unit 105 uses an image signal for each pixel at the distance L₀ received from the radiation detector 102 to generate three-dimensional dose distribution information of the dose of radiation emitted from the radiation irradiation unit 101. An image signal D_{0,i,j} for each pixel of the radiation detector 102 detected at the distance L₀ is converted into an image signal D_{1,i,j} for each pixel at the distance Lᵢ by:

$${D}_{1 , i , j} ( {x}_{1 , i , j} , {y}_{1 , i , j} , {L}_{1} , θ ) = \frac{( {x}_{0 , i , j}^{2} + {y}_{0 , i , j}^{2} + {L}_{0}^{2} )}{\begin{matrix}{\{ \frac{{L}_{1}}{( {L}_{0} - {x}_{0 , i , j} tan θ )} \cdot {x}_{0 , i , j} \}}^{2} + \\ {\{ \frac{{L}_{1}}{( {L}_{0} - {x}_{0 , i , j} tan θ )} \cdot {y}_{0 , i , j} \}}^{2} + {\{ \frac{{L}_{1}}{( {L}_{0} - {x}_{0 , i , j} tan θ )} \cdot {L}_{0} \}}^{2}\end{matrix}} \times {D}_{0. i . j} ( {x}_{0 , i , j} , {y}_{0 , i , j} , {L}_{0} ) = \frac{{( {L}_{0} - {x}_{0 , i , j} tan θ )}^{2}}{{L}_{1}^{2}} \times {D}_{0 , i , j} ( {x}_{0 , i , j} , {y}_{0 , i , j} , {L}_{0} ) \qquad ( g )$$

The difference between equations (b) and (g)is that even if a deviation of the angle θ occurs, three-dimensional dose distribution information of the radiation dose on the plane at the distance L₁ can be calculated by equation (g).

The image processing unit 105 approximates, by a quadratic function given by equation (c), the image signal D_{1,i,j} given by equation (g), thereby deciding each coefficient of the quadratic function by the least square method. Then, with respect to all the pixels, the image processing unit 105 divides, by dose distribution information at a corresponding pixel position, an image signal S_{2,i,j} for each pixel received from the radiation detector 102 by radiation imaging, as given by equation (f). At this time, in the processing of this embodiment, the coefficients of the quadratic function decided from equation (g) are used. The image signal S_{2,i,j} for each pixel received from the radiation detector 102 includes the variation of the image signal caused by the three-dimensional dose distribution regarding the radiation emitted from the radiation irradiation unit 101, that is, the variation of the image signal that may be caused in accordance with the three-dimensional distance relationship including the relative angle deviation between the radiation irradiation unit 101 and the radiation detector 102. By dividing the image signal S_{2,i,j} by the three-dimensional dose distribution information, the image signal (output signal) of each pixel of the radiation detector 102 is corrected based on the three-dimensional distance relationship between the radiation irradiation unit 101 and the radiation detector 102.

### (Modification)

Note that in the second embodiment, a threshold may be set for the difference between the pieces of three-dimensional dose distribution information on the planes at the distances L₀ and L₁. If the difference is equal to or smaller than the threshold, the dose distribution information need not be corrected, and only if the difference exceeds the threshold, the dose distribution correction processing in step S504 may be performed. In the second embodiment, in accordance with a request by the operator, whether to perform the dose distribution correction processing in step S504 may be switched.

According to this embodiment, if a relative angle deviation between the radiation irradiation unit 101 and the radiation detector 102 occurs, it is possible to reduce the variation of the image signal that may be caused in accordance with the three-dimensional positional relationship including the relative angle deviation between the radiation irradiation unit 101 and the radiation detector 102, and suppress non-uniformity of an image that may be caused by the three-dimensional dose distribution regarding the radiation. This can reduce the variation of the image signal with high accuracy, as compared with the first embodiment, even if a relative angle deviation between the radiation irradiation unit 101 and the radiation detector 102 occurs, and can suppress non-uniformity of the image that may be caused by the three-dimensional dose distribution regarding the radiation.

### Third Embodiment

The third embodiment of the present disclosure will be described next. This embodiment is different from the first embodiment in that even when a radiation detector 102 images an object P while rotating about the irradiation center axis of a radiation irradiation unit 101 at the time of imaging the object, the variation of an image signal that may be caused in accordance with a three-dimensional positional relationship is reduced.

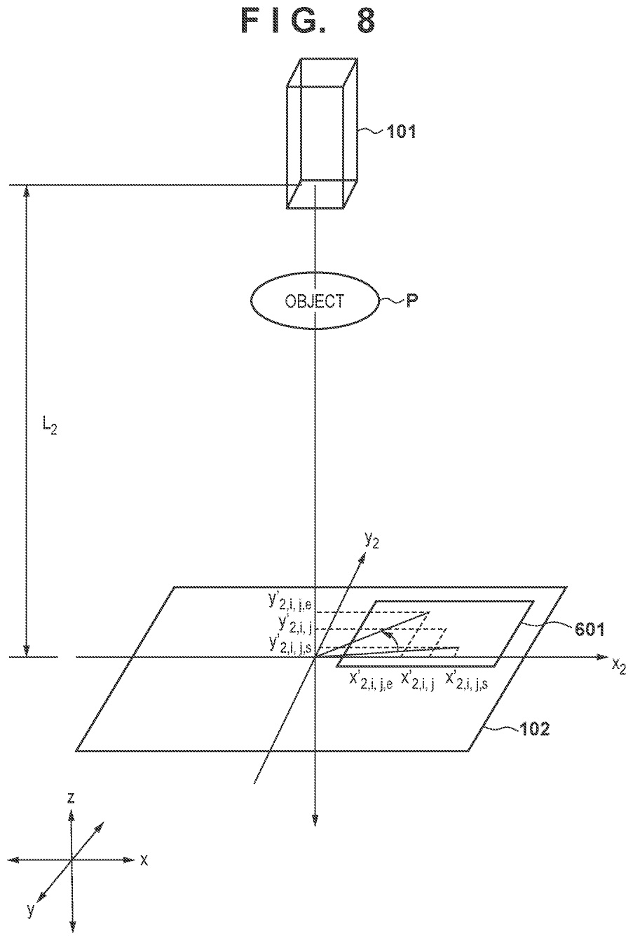

The same description as in the first embodiment will be omitted, and only the difference from the first embodiment will be described below. Processing of generating three-dimensional dose distribution information of a radiation dose according to the third embodiment will be described with reference to FIG. 8.

FIG. 8 is a view exemplifying the positional relationship between the radiation irradiation unit 101 and the radiation detector 102 in the processing of generating the three-dimensional dose distribution information. In the third embodiment, an imaging control unit 104 includes a rotation mechanism for rotating the radiation detector 102 about an irradiation center axis 801 of the radiation irradiation unit 101, and can control the rotation mechanism based on predetermined imaging condition information. In the processing in step S502 of FIG. 5, the imaging control unit 104 controls the rotation mechanism at the time of radiation irradiation to rotate the radiation detector 102.

At the time of imaging the object, when the radiation detector 102 is relatively moved with respect to the radiation irradiation unit 101, an image processing unit 105 averages dose distribution information obtained at a position before the movement and that obtained at a position after the movement, thereby obtaining three-dimensional dose distribution information for correcting an image signal.

As shown in FIG. 8, in the third embodiment, in the processing in step S502, at the time of radiation irradiation, a pixel of the radiation detector 102 is moved from X′_{2,i,j,s} before the movement to X′_{2,i,j,s} after the movement in the X-axis direction, and is moved from Y′_{2,i,j,s} before the movement to Y′_{2,i,j,s} after the movement in the Y-axis direction.

Image signal correction processing according to the third embodiment is performed by division processing using equation (h) below in the dose distribution correction processing in step S504 of FIG. 5 according to the first embodiment.

$${P}_{2 , i , j} ( {x}_{2 , i , j}^{′} , {y}_{2 , i , j}^{′} , {L}_{2} , {x}_{2 , i , j , s}^{′} , {y}_{2 , i , j , s}^{′} , {x}_{2 , i , j , e}^{′} , {y}_{2 , i , j , e}^{′} , ) = \frac{{S}_{2 , i , j} ( {x}_{2 , i , j}^{′} , {y}_{2 , i , j}^{′} , {L}_{2} )}{\begin{matrix}\frac{1}{2} {a}_{2} ( {x}_{2 , i , j , s}^{′ 2} + {x}_{2 , i , j , e}^{′ 2} ) + \frac{1}{2} {b}_{2} ( {x}_{2 , i , j , s}^{′} + {x}_{2 , i , j , e}^{′} ) + \\ \frac{1}{2} {c}_{2} ( {y}_{2 , i , j , s}^{′ 2} + {y}_{2 , i , j , e}^{′ 2} ) + \frac{1}{2} {d}_{2} ( {y}_{2 , i , j , s}^{′} + {y}_{2 , i , j , e}^{′} ) + {e}_{2}\end{matrix}} \qquad ( h )$$

In equation (h), a quadratic function in the denominator is based on equation (f), and pieces of dose distribution information at the coordinates (X′_{2,i,j,s}, Y′_{2,i,j,s}) of the start point of the movement of the pixel of the radiation detector 102 and the coordinates (X′_{2,i,j,s}, Y′_{2,i,j,s}) of the end point of the movement are averaged.

In radiation imaging performed along with rotation, as shown in FIG. 8, with respect to all the pixels, the image processing unit 105 divides an image signal S_{2,i,j} for each pixel received from the radiation detector 102 by the averaged dose distribution information at the corresponding pixel position, as given by equation (h).

With the processing using equation (h), it is possible to correct the three-dimensional dose distribution information by almost averaging the radiation doses of the radiation irradiation unit 101 within a rotation range at the time of imaging the object, and correct the image signal using the corrected three-dimensional dose distribution information.

### (Modification)

Note that in the third embodiment, in the processing of averaging the pieces of dose distribution information at the start and end points of the movement of the pixel of the radiation detector 102, the line integral of the dose distribution information may be obtained within the movement range, and division processing may be performed by a length with which the line integral is obtained. Instead of averaging the pieces of dose distribution information by calculation, dose distribution information may be generated by obtaining an image signal for each pixel in a state in which the radiation detector 102 is rotated about the irradiation center axis of the radiation irradiation unit 101 in steps S205 to S207 of FIG. 2.

In the third embodiment, the rotation speed of the radiation detector 102 may be changed in accordance with the type of the object P and the purpose of object imaging. If the operator inspects the fine structure of the object P, the rotation speed may be decreased. If the operator wants to improve the efficiency of actual imaging, the rotation speed may be increased.

The third embodiment has explained the example in which the radiation detector 102 images the object P while rotating about the rotation axis (z-axis) of the radiation irradiation unit 101 at the time of imaging the object. The arrangement of the radiation imaging apparatus is not limited to this. For example, at least two of the radiation irradiation unit 101 for emitting radiation, a holder (holding stage) for holding the object P, and the radiation detector 102 for detecting the radiation are configured to be movable (for example, rotatable in synchronism with each other) within a plane intersecting the rotation axis (z-axis).

At this time, at least two of the above units are configured to be movable (for example, rotatable in synchronism with each other) within the plane intersecting the rotation axis so as to satisfy the positional relationship in which the radiation emitted from the radiation irradiation unit 101 is transmitted through the object P in a direction inclined with respect to the rotation axis and can be detected by the radiation detector 102. For example, the radiation irradiation unit 101, the holder (holding stage), and the radiation detector 102 may be configured to be movable within the plane intersecting the rotation axis so as to satisfy the above positional relationship. Alternatively, for example, the radiation irradiation unit 101 and the radiation detector 102 may be configured to be movable within the plane intersecting the rotation axis in a state in which the holder (holding stage) stops at the position of the rotation axis so as to satisfy the above positional relationship. Alternatively, for example, the radiation irradiation unit 101 and the holder (holding stage) may be configured to be movable within the plane intersecting the rotation axis in a state in which the radiation detector 102 stops at the position of the rotation axis so as to satisfy the above positional relationship.

According to this embodiment, even if the radiation detector 102 images the object P while rotating about the irradiation center axis of the radiation irradiation unit 101 at the time of imaging the object, it is possible to reduce the variation of an image signal that may be caused in accordance with the three-dimensional positional relationship between the radiation irradiation unit 101 and the radiation detector 102, and suppress non-uniformity of an image that may be caused by the three-dimensional dose distribution regarding the radiation. This can reduce the variation of the image signal with high accuracy, as compared with the first embodiment, even if the radiation detector 102 rotates, and can suppress non-uniformity of the image that may be caused by the three-dimensional dose distribution regarding the radiation.

### Fourth Embodiment

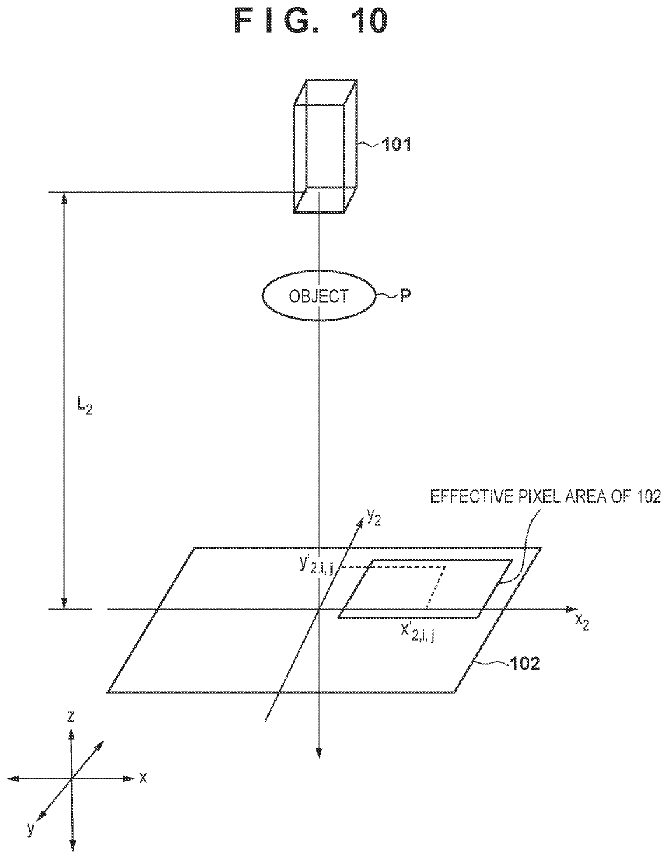

The fourth embodiment of the present disclosure will be described next. This embodiment is different from the first embodiment in that three-dimensional dose distribution information of a radiation dose is monitored. The same description as in the first embodiment will be omitted, and only the difference from the first embodiment will be described below. FIG. 10 is a view exemplifying the positional relationship between a radiation irradiation unit 101 and a radiation detector 102 in processing according to the fourth embodiment. This embodiment will describe an arrangement for executing each process below.

### (Notification of Pixel Region Not to Be Used at Time of Imaging Object)

An arrangement for notifying an operator of a pixel region of the radiation detector 102 not to be used at the time of imaging an object as a result of monitoring dose distribution information will be described. In the fourth embodiment, a processing procedure of generating dose distribution information shown in FIG. 2 is performed at a preset timing (for example, once a month or the like).

In this embodiment, an image display unit 106 (display control unit) displays information obtained from an image processing unit 105 on a display device. The image processing unit 105 obtains position information of a pixel of the radiation detector 102, whose image signal exceeds a threshold (dose threshold), and the image display unit 106 (display control unit) converts the position information of the pixel into an image on a plane on which dose distribution information is obtained, and displays the image on the display device.

In step S207 of FIG. 2, the image processing unit 105 determines, for the image signal for each pixel obtained on a plane at a distance L₀ and received in step S206, whether the image signal is equal to or smaller than the predetermined threshold. If the image signals for all the pixels are equal to or smaller than the threshold, this processing ends. On the other hand, if there exists at least one pixel whose image signal exceeds the threshold, the image processing unit 105 determines (obtains) the position of the corresponding pixel on the plane at a distance L₁ or L₂ with respect to the pixel whose image signal exceeds the threshold on the plane at the distance L₀. The image processing unit 105 obtains the position of the pixel, within the plane at the distance L₁ or L₂, corresponding to the position of the pixel whose image signal exceeds the threshold on the plane at the distance L₀ using the positional relationship (the positional relationship based on the distance ratio) between the plane at the distance L₀ and the plane at the distance L₁ or L₂. Note that if there exist a plurality of pixels whose image signals exceed the threshold, the image processing unit 105 obtains the positions of a plurality of pixel regions where the image signals exceed threshold. Then, the image processing unit 105 transmits the obtained pixel position information to the image display unit 106.

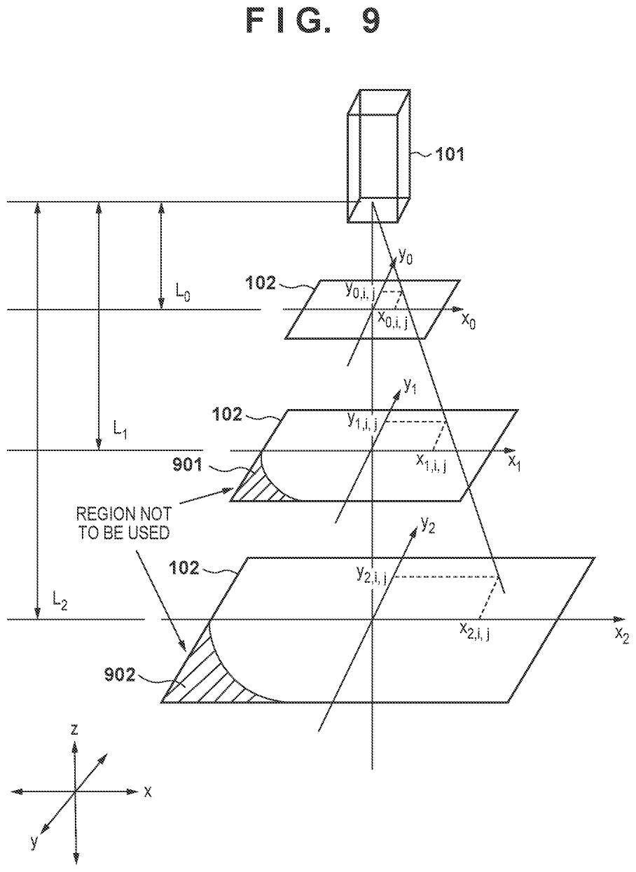

The image display unit 106 converts the received pixel position information into an image on the plane at the distance L₁ or L₂, and outputs (displays) the converted image to the display device such as a monitor. FIG. 9 is a view showing an example of display of a region not to be used in radiation imaging. Referring to FIG. 9, a region 901 indicates the position of a pixel (pixel region) whose image signal exceeds the threshold on the plane at the distance L₁, and a region 902 indicates the position of a pixel (pixel region) whose image signal exceeds the threshold on the plane at the distance L₂. Each of the regions 901 and 902 is a pixel region where the variation of the image signal exceeds the threshold, and the image display unit 106 presents, to the operator, the region as a region not to be used in radiation imaging, as shown in FIG. 9. Note that the presenting method is not limited to the display of the image, as indicated by the region 901 or 902. The received pixel position information may be converted into a character string to present pixel coordinates (pixel position). Both the pieces of information may be presented in combination.

With the above processing, it is possible to notify the operator of a region not to be used at the time of imaging the object as a result of monitoring the dose distribution information. By confirming the region (region 901 or 902) shown in FIG. 9 before radiation imaging, the operator can perform imaging by setting an effective pixel area not to include such region. This can reduce the variation of the image signal, and suppress non-uniformity of an image that may be caused by a three-dimensional dose distribution regarding radiation.

### (Notification of Presence/Absence of Temporal Change of Image Signal)

An arrangement for notifying the operator of the result of monitoring a temporal change of dose distribution information and determining whether the temporal change of the image signal is normal will be described next. The image processing unit 105 determines whether a temporal change of dose distribution information obtained based on a plurality of pieces of dose distribution information obtained at different timings is equal to or smaller than a temporal threshold, and the image display unit 106 (display control unit) displays the determination result of the image processing unit 105 on the display device.

In the fourth embodiment, in step S207 of the second or subsequent processing procedure (FIG. 2) of generating dose distribution information, the image processing unit 105 calculates (obtains) the time differential value of each coefficient of a quadratic function representing the dose distribution information based on:

$$\{ \begin{matrix}\frac{{da}_{2}}{dt} = \frac{{a}_{2 , 2} - {a}_{2 , 1}}{T} \\ \frac{{db}_{2}}{dt} = \frac{{b}_{2 , 2} - {b}_{2 , 1}}{T} \\ \frac{d {c}_{2}}{dt} = \frac{{c}_{2 , 2} - {c}_{2 , 1}}{T} \\ \frac{{dd}_{2}}{dt} = \frac{{d}_{2 , 2} - {d}_{2 , 1}}{T} \\ \frac{{de}_{2}}{dt} = \frac{{e}_{2 , 2} - {e}_{2 , 1}}{T}\end{matrix} \qquad ( i )$$

In equations (i), coefficients a_{2,1}, b_{2,1}, c_{2,1}, d_{2,1}, and e_{2,1} are the respective coefficients of the quadratic function representing the dose distribution information on the plane at the distance L₂ calculated (obtained) in the preceding processing procedure of generating the dose distribution information. Furthermore, in equations (i), coefficients a_{2,2}, b_{2,2}, c_{2,2}, d_{2,2}, and e_{2,2} are the respective coefficients of the quadratic function representing the dose distribution information on the plane at the distance L₂ calculated (obtained) in the this (current) processing procedure of generating the dose distribution information. Furthermore, T represents a time interval between the time of performing the preceding processing procedure of generating the dose distribution information and the time of performing this (current) processing procedure of generating the dose distribution information.

The image processing unit 105 determines whether the absolute value of the time differential value of each coefficient calculated (obtained) by equations (i) falls within a predetermined threshold range, and transmits determination result information to the image display unit 106. The image display unit 106 converts the received determination information into a character string, and outputs (displays) the converted character string to the display device such as a monitor. If the absolute value of the time differential value of each coefficient falls within the predetermined threshold range, information indicating the normal state is presented to the operator. On the other hand, if the absolute value of the time differential value of each coefficient falls outside the predetermined threshold range, information indicating the abnormal state is presented to the operator. Note that the temporal change of the radiation dose distribution information of a radiation irradiation apparatus may be monitored, and only if a change amount exceeds a reference value, the operator may be notified of it.

### (Prediction of Failure Timing)

An arrangement for monitoring a temporal change of dose distribution information and predicting, based on the temporal change of the dose distribution information, a timing at which the radiation irradiation unit 101 fails will be described next.

In the fourth embodiment, the image processing unit 105 calculates the difference between a predetermined threshold and the three-dimensional dose distribution information (each coefficient of the quadratic function) on the plane at the distance L₂ calculated in the current processing procedure of generating the dose distribution information. The predetermined threshold (failure determination threshold) indicates a failure determination criterion. The difference value between the dose distribution information and the threshold indicates a margin of the dose distribution before a failure occurs. The image processing unit 105 can estimate a failure timing based on this (current) dose distribution information by dividing the difference value by the temporal change of the dose distribution information (the time differential value of each coefficient calculated by equations (i)). That is, the image processing unit 105 according to this embodiment estimates the failure timing of the radiation irradiation unit 101 based on the information (quotient information) obtained by dividing the difference value between the dose distribution information and the failure determination threshold by the temporal change of the dose distribution information.

The image processing unit 105 transmits the calculated quotient information to the image display unit 106. The image display unit 106 (display control unit) displays the estimation result of the image processing unit 105 on the display device. The image display unit 106 converts the received quotient information into numerical value information, and outputs (displays) the converted numerical value information on the display device such as a monitor. The numerical value information output (displayed) by the image display unit 106 indicates prediction information before the radiation irradiation unit 101 fails, and the operator can grasp the predicted timing at which the radiation irradiation unit 101 fails by confirming the numerical value information.

With this processing, it is possible to predict, by monitoring the temporal change of the dose distribution information, a timing at which the radiation irradiation unit 101 fails.

This allows the operator to select an imaging enable position even if there is a sign of a failure of the radiation irradiation unit 101. Furthermore, the operator can recognize a failure of the radiation irradiation unit 101, thereby reducing the risk of ineffectively applying radiation to the object P and performing imaging again. The operator can recognize the predicted timing at which the radiation irradiation unit 101 fails, and replace the radiation irradiation unit 101 before the radiation irradiation unit 101 fails.

### (Processing of Monitoring and Correcting Temporal Change of Dose Distribution Information)

Processing of monitoring a temporal change of dose distribution information, predicting, based on the temporal change, the dose distribution information of the radiation irradiation unit 101 at the time of imaging the object, and correcting the dose distribution information based on the prediction result will be described next. In this embodiment, the image processing unit 105 corrects the dose distribution information using information indicating the temporal change, and corrects, using the corrected dose distribution information, an image signal output from the radiation detector 102 at the time of imaging the object.

In the fourth embodiment, in the correction processing in step S504 after performing the second or subsequent processing procedure of generating the dose distribution information, the image processing unit 105 divides the image signal S_{2,i,j} for each pixel by the dose distribution information at the corresponding pixel position based on:

$${P}_{2 , i , j} ( {x}_{2 , i , j}^{′} , {y}_{2 , i , j}^{′} , {L}_{2} , {T}^{′} ) = \frac{{S}_{2 , i , j} ( {x}_{2 , i , j}^{′} , {y}_{2 , i , j}^{′} , {L}_{2} )}{\begin{matrix}( {a}_{2 , 2} + {T}^{′} \times \frac{{da}_{2}}{dt} ) {x}_{2 , i , j}^{′ 2} + ( {b}_{2 , 2} + {T}^{′} \times \frac{{db}_{2}}{dt} ) {x}_{2 , i , j}^{′} + \\ \begin{matrix}( {c}_{2 , 2} + {T}^{′} \times \frac{d {c}_{2}}{dt} ) {y}_{2 , i , j}^{′ 2} + ( {d}_{2 , 2} + {T}^{′} \times \frac{{dd}_{2}}{dt} ) {y}_{2 , i , j}^{′} + \\ {e}_{2 , 2} + {T}^{′} \times \frac{{de}_{2}}{dt}\end{matrix}\end{matrix}} \qquad ( j )$$

where T′ represents a time interval from when the immediately preceding (previous) processing procedure of generating the dose distribution information is performed until this (current) actual imaging processing is performed. In equation (j), each coefficient of the dose distribution information in the denominator includes a temporal change of each coefficient obtained by equations (i), and each coefficient of the quadratic function and a value obtained by multiplying the time differential value of each coefficient by the time interval T′ are added. This can correct the three-dimensional dose distribution information using the temporal change of the three-dimensional dose distribution information regarding the radiation emitted from the radiation irradiation unit 101, and the image signal (output signal) of each pixel of the radiation detector 102 is corrected based on the three-dimensional distance relationship between the radiation irradiation unit 101 and the radiation detector 102 by dividing the image signal S_{2,i,j} for each pixel by the corrected dose distribution information.

With this processing, it is possible to monitor a temporal change of the dose distribution information, predict, based on the temporal change, the dose distribution information of the radiation irradiation unit 101 at the time of imaging the object, and correct the dose distribution information based on the prediction result.

In each process described above, the processing procedure of generating the dose distribution information described with reference to FIG. 2 may be performed every time the radiation imaging apparatus 100 is activated or performed at a predetermined timing.

According to this embodiment, it is possible to reduce the variation of an image signal that may be caused in accordance with the three-dimensional positional relationship between the radiation irradiation unit 101 and the radiation detector 102. Furthermore, it is possible to correct the dose distribution information that can temporally change, and correct the image signal using the corrected three-dimensional dose distribution information. This can suppress non-uniformity of the image that may be caused by the three-dimensional dose distribution regarding the radiation.

According to the present disclosure, it is possible to reduce the variation of the image signal that may be caused in accordance with the three-dimensional positional relationship between the radiation irradiation apparatus and the detector.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processing units (e.g., central processing unit (CPU), micro processing unit (MPU)) and may include a network of separate computers or separate processing units to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-183448, filed Nov. 10, 2021, which is hereby incorporated by reference herein in its entirety.

What is claimed is:

1. A radiation imaging apparatus including a detection unit configured to detect radiation emitted from a radiation irradiation unit, the apparatus comprising:
a processing unit configured to obtain dose distribution information regarding the radiation with which the detection unit is irradiated from the radiation irradiation unit,
wherein the processing unit corrects, using the dose distribution information, an image signal output from the detection unit.

2. The apparatus according to claim 1, wherein the processing unit obtains the dose distribution information using a distance relationship between the detection unit and the radiation irradiation unit.

3. The apparatus according to claim 1, wherein the processing unit obtains the dose distribution information in accordance with a first distance relationship between the detection unit and the radiation irradiation unit and a second distance relationship differing from the first distance relationship.

4. The apparatus according to claim 1, wherein
the processing unit obtains the dose distribution information in accordance with a plurality of imaging conditions set in the radiation irradiation unit, and
the plurality of imaging conditions include at least one of a tube voltage, a tube current, an irradiation time, a focal size, and a radiation filtration filter.

5. The apparatus according to claim 1, wherein the processing unit switches the dose distribution information to be used for the correction in accordance with an imaging condition selected from a plurality of imaging conditions.

6. The apparatus according to claim 1, wherein the processing unit corrects the image signal using dose distribution information for each of a plurality of regions obtained by dividing a detection surface of the detection unit.

7. The apparatus according to claim 1, wherein the processing unit corrects, using the dose distribution information obtained based on the image signal of the detection unit, an image signal output from a different detection unit.

8. The apparatus according to claim 1, wherein
the processing unit specifies, from an image signal at the time of imaging an object, a through-exposure region as a region where the detection unit is directly irradiated with the radiation, and
the processing unit corrects the dose distribution information using an image signal of the through-exposure region.

9. The apparatus according to claim 1, wherein if an image signal output from each pixel of the detection unit changes to exceed a threshold, the processing unit corrects the dose distribution information using an image signal for which sensitivity has been corrected not to exceed the threshold.

10. The apparatus according to claim 1, wherein the processing unit
corrects the dose distribution information using an angle relationship between the detection unit and the radiation irradiation unit, detected at the time of imaging an object, and
corrects the image signal using the corrected dose distribution information.

11. The apparatus according to claim 1, wherein if the detection unit is relatively moved with respect to the radiation irradiation unit at the time of imaging an object, the processing unit obtains dose distribution information for correcting the image signal by averaging dose distribution information obtained at a position before the movement and dose distribution information obtained at a position after the movement.

12. The apparatus according to claim 1, wherein at least two of a radiation irradiation unit configured to emit the radiation, a holding unit configured to hold an object, and a detection unit configured to detect the radiation are configured to be movable within a plane intersecting a rotation axis so as to satisfy a positional relationship in which the radiation emitted from the radiation irradiation unit is transmitted through the object in a direction inclined with respect to the rotation axis and can be detected by the detection unit.

13. The apparatus according to claim 1, further comprising a display control unit configured to display, on a display unit, information obtained from the processing unit,
wherein the processing unit obtains position information of a pixel of the detection unit, whose image signal exceeds a threshold, and
the display control unit converts the position information of the pixel into an image on a plane on which the dose distribution information is obtained, and displays the image on the display unit.

14. The apparatus according to claim 13, wherein the processing unit determines whether a temporal change of dose distribution information obtained based on a plurality of pieces of dose distribution information obtained at different timings does not exceed a temporal threshold, and
the display control unit displays a result of the determination on the display unit.

15. The apparatus according to claim 14, wherein the processing unit corrects the dose distribution information using information indicating the temporal change, and corrects, using the corrected dose distribution information, the image signal output from the detection unit at the time of imaging an object.

16. The apparatus according to claim 13, wherein the processing unit estimates a failure timing of the radiation irradiation unit from information obtained by dividing a difference between the dose distribution information and a failure determination threshold by a temporal change of the dose distribution information, and
the display control unit displays a result of the estimation on the display unit.

17. An image processing apparatus comprising a processing unit configured to correct, using three-dimensional dose distribution information regarding radiation emitted from a radiation irradiation unit, an image signal output from a detection unit configured to detect the radiation.

18. An operation method for a radiation imaging apparatus including a detection unit configured to detect radiation emitted from a radiation irradiation unit, comprising:
obtaining dose distribution information regarding the radiation with which the detection unit is irradiated from the radiation irradiation unit; and
correcting, using the dose distribution information, an image signal output from the detection unit.

19. A non-transitory computer readable storage medium storing a program for causing a computer to execute an operation method defined in claim 18.
